(19) United States
(12) Patent Application Publication
(10) Pub. No.: US 2024/0002308 A1
(43) Pub. Date: Jan. 4, 2024

(54) SOIL-STRUCTURE IMPROVING BIO-ORGANIC FERTILIZER AND PREPARATION METHOD THEREOF

(71) Applicants: Binzhou Jingyang biological fertilizer Co., Ltd, Binzhou (CN); Shandong Academy of Agricultural Sciences, Jinan (CN)

(72) Inventors: Lihua Jiang, Jinan (CN); Chunxiu Wei, Jinan (CN); Yan Yang, Jinan (CN); Baozhong Zhao, Jinan (CN); Xueping Li, Jinan (CN); Lesen Zhang, Jinan (CN); Yu Xu, Jinan (CN); Jing Shi, Jinan (CN); Mei Wang, Jinan (CN); Yumin Liu, Jinan (CN)

(21) Appl. No.: 18/466,994

(22) Filed: Sep. 14, 2023

Related U.S. Application Data
(63) Continuation of application No. PCT/CN2022/102712, filed on Jun. 30, 2022.

(30) Foreign Application Priority Data
May 30, 2022 (CN) 202210603367.7

(51) Int. Cl. C05F 17/50; C12N 1/20; C12P 1/04; C05F 11/08

(52) U.S. Cl. CPC C05F 17/50; C12N 1/20; C12P 1/04; C05F 11/08

(57) ABSTRACT

The present invention discloses a soil-structure improving bio-organic fertilizer and a preparation method thereof, and belongs to the technical field of biological fertilizers. The preparation method of the bio-organic fertilizer of the present invention includes the following steps: S1: aerobically fermenting cow dung-based organic materials to prepare a solid-state fermentation medium; S2: inoculating Bacillus subtilis fermentation broth and silicate bacteria fermentation broth into the solid-state fermentation medium for solid-state fermentation until the end of fermentation; and S3: after the fermentation is finished, extruding and granulating or directly sieving to prepare the soil-structure improving bio-organic fertilizer.

### TECHNICAL FIELD

The present invention relates to the technical field of biological fertilizers, in particular to a soil-structure improving bio-organic fertilizer and a preparation method thereof.

### BACKGROUND

Microbial fertilizer refers to a product with effects of both microbial fertilizer and organic fertilizer, which is formed by compounding organic materials obtained by harmless treatment and decomposition with specific functional microorganisms and animal and plant residues (such as livestock manure and crop straws) as sources. The microbial fertilizer can improve the physical and chemical properties of soil, improve the micro-ecosystem of soil, enhance the fertility level of soil, increase the crop yield, improve the crop quality, and reduce or decrease the occurrence of plant diseases and pests.

At present, the research and development of microbial fertilizer in China focuses on plant growth promoting rhizobacteria and microbial fertilizer strains capable of repairing soil. At this stage, types and functions of microbial fertilizer products are gradually diversified; and multi-strain compound biological fertilizers gradually become the mainstream. In terms of product composition, functional microbial agents are mixed with carrier materials; various functional microbial agents are mixed with chemical fertilizers; and especially in the aspect of compounding functional microorganisms and organic and inorganic nutrients (functional microbial organic fertilizer), a series of products are developed.

However, microbial fertilizers in the prior art are mostly produced by such a manner that microbial strain liquid is fermented and then adsorbed and aged with carriers to prepare the microbial fertilizers. Therefore, the carriers cannot be organically combined with the functional microorganisms; the effects of the microbial fertilizers obtained by mixing are unstable; and the characteristics of the functional strains cannot be fully exerted. In addition, for example, patent 202010283878.6-Solid State Fermentation Production Method of Microbial Fertilizer and Solid Compound Microbial Fertilizer, relates to a compound microbial fertilizer, which has high content of viable bacteria, has a main function of improving the disease resistance and disease prevention ability of crops, but takes livestock manure and seed meal as adsorption substrates, thereby having relatively high nutrient content, mainly playing a role of providing nutrients, and having poor effect of improving the soil structure.

Therefore, it is an urgent problem for those skilled in the art to develop a bio-organic fertilizer capable of organically combining carriers with functional microorganisms, realizing stable fertilizer effect, giving full play to the characteristics of functional strains and having a better effect of improving the soil structure.

### SUMMARY

In view of this, the present invention provides a soil-structure improving bio-organic fertilizer and a preparation method thereof. Cow dung and plant straws with higher cellulose content are taken as raw materials for aerobic fermentation to obtain a solid medium, thereby increasing the content of cellulose in the solid medium and having a good effect on improving the soil structure. Bacillus subtilis and silicate bacteria are inoculated on the solid medium for solid-state fermentation culture, so that the solid fermentation substrate is organically combined with the functional strains, thereby solving the problems of failure in organic combination of carriers and the functional microorganisms and unstable effect of microbial fertilizers obtained by mixing, giving full play to characteristics of the functional bacteria, and improving the the number and viability of strains in the bio-organic fertilizer. Meanwhile, silicate bacteria produce a large number of polysaccharides, which have aggregate structures and have a good effect on improving the soil structure.

To achieve the above purpose, the following technical solution is adopted by the present invention:

A preparation method of a soil-structure improving bio-organic fertilizer includes the following steps:

S1: aerobically fermenting cow dung-based organic materials to obtain a solid-state fermentation medium;

S2: mixing and inoculating Bacillus subtilis fermentation broth and silicate bacteria fermentation broth into the solid-state fermentation medium obtained in S1 for solid-state fermentation;

S3: after the fermentation is finished, extruding and granulating or directly sieving to prepare the soil-structure improving bio-organic fertilizer.

Preferably, the cow dung-based organic materials are composed of any of the following three types in parts by weight:
1) 50-60 parts of cow dung, 40-50 parts of tomato straw and 5-10 parts of moisture regulating auxiliary materials;
2) 50-60 parts of cow dung, 40-50 parts of corn straw and 5-10 parts of moisture regulating auxiliary materials;
3) 40-50 parts of cow dung, 20-30 parts of tomato straw, 15-20 parts of corn straw and 5-10 parts of moisture regulating auxiliary materials.

Preferably, the moisture regulating auxiliary materials are one or two of sugar residues or rice husks.

Further preferably, in the present invention, 50-60 parts of cow dung, 40-50 parts of tomato straw, 5-10 parts of moisture regulating auxiliary materials and 0.5% of commercially available decomposing agent are used for aerobic fermentation to obtain a solid-state fermentation medium. The technical indexes by weight percentage include: 35%±5% of moisture content, 40%±5% of organic matter, 1.3%±0.3% of total nitrogen, 1.3%±0.3% of P₂O₅ and 1.6%±0.3% of K₂O.

Further preferably, in the present invention, 50-60 parts of cow dung, 40-50 parts of corn straw, 5-10 parts of moisture regulating auxiliary materials and 0.5% of commercially available decomposing agent are used for aerobic fermentation to obtain a solid-state fermentation medium. The technical indexes by weight percentage include: 30%±5% of moisture content, 45%±5% of organic matter, 1.2%±0.3% of total nitrogen, 1.2%±0.3% of P₂O₅ and 1.5%±0.3% of K₂O.

Further preferably, in the present invention, 40-50 parts of cow dung, 20-30 parts of tomato straw, 15-20 parts of corn straw, 5-10 parts of moisture regulating auxiliary materials and 0.5% of commercially available decomposing agent are used for aerobic fermentation to obtain a solid-state fermentation medium. The technical indexes by weight percentage include: 32%±5% of moisture content, 42%±5% of organic matter, 1.2%±0.4% of total nitrogen, 1.2%±0.4% of P₂O₅ and 1.5%±0.4% of K₂O.

Preferably, the cow dung-based organic materials has an initial moisture content of 50-55%; and after the temperature of the pile reaches 55° C., the fermentation is continued for 7-10 days, and then the fermentation is finished.

Preferably, the cow dung-based organic materials are stacked according to the length×width×height of 50 m×5 m×1.5 m. During the whole period of aerobic fermentation, a pile is turned over for 3-4 times; and when the temperature of the pile is higher than 65° C., the temperature is lowered by turning over, stirring and aeration.

The above technical solution has the beneficial effects that: when the temperature of the pile is above 55° C., the fermentation is continued for 7-10 days, to ensure that the mortality rate of ascaris eggs, colititer and other sanitary indicators are up to the standard; during the whole aerobic period, the pile is turned over for 3-4 times, thereby facilitating the pile to be in full contact with oxygen and accelerating the fermentation and decomposition of materials; and when the temperature of the pile is higher than 65° C., the temperature is reduced by turning over, stirring and aeration, to avoid excessive temperature and nutrient loss of carbon and nitrogen.

Preferably, the inoculation weights of the Bacillus subtilis fermentation broth and the silicate bacteria fermentation broth to the solid-state fermentation medium are 5-15% of the weight of the solid-state fermentation medium, respectively.

The above technical solution has the beneficial effects that: Bacillus subtilis and silicate bacteria are co-fermented to realize a synergistic effect, so that in a first aspect, the number of viable silicate bacteria is increased; in a second aspect, the two bacteria are fermented together to improve the total number of viable bacteria in the fertilizer; and in a third aspect, the silicate bacteria produce a large number of polysaccharides, which is conducive to the formation of a soil aggregate structure and can effectively improve the soil structure; and the Bacillus subtilis can improve the stress resistance of plants.

Preferably, the solid fermentation materials have an initial moisture content of the pile is turned over every 22-26 hours during fermentation; and the fermentation time is 5-7 days.

Preferably, the solid fermentation materials are stacked according to the length×width×height of 50 m×5 m×0.5 m. The solid fermentation materials have a fineness of meshes, a C/N ratio of 25-30, and a pH of 6.5-7.5.

Preferably, the solid fermentation materials have the fineness of 80 meshes, the C/N ratio of 28, and the pH of 7.

The above technical solution has beneficial effects that: the initial materials in the present invention have the moisture content of 40-50%, which enables the materials to absorb water, expand and soften, is conducive to the decomposition of microorganisms, and provides a basis for the organic combination of the microorganisms and the solid fermentation materials; the manner of stacking and turning over ensures the heat loss in fermentation and the content of oxygen in the fermentation materials; the defined fineness of materials is conducive to the uniform mixing of solid materials and microbial broth, the acceleration of fermentation, and the organic combination of microbial broth and solid substrate; and the defined C/N ratio and pH of the materials ensure the carbon-nitrogen balance in the compost mixture, increase the number of viable bacteria in the fermented product, promote the further development of the potential of the functional strains, and enable the microorganisms to achieve the optimal biological activity.

Preferably, a preparation method of the Bacillus subtilis fermentation broth comprises: inoculating the Bacillus subtilis broth obtained by shake-flask culture into a seed tank culture medium according to an inoculation amount of 2-5% of the mass of the seed tank culture medium, and culturing for 22-26 hours at 28-32° C. and 180-200 rpm, with a ventilation of 0.9 (V/V·min), to obtain the Bacillus subtilis fermentation broth.

A preparation method of the silicate bacteria fermentation broth comprises: inoculating the silicate bacteria broth obtained by shake-flask culture into a seed tank culture medium according to the inoculation amount of 4-6% of the mass of the seed tank culture medium, and culturing for 28-32 hours at 28-32° C. and 180-220 rpm, with a ventilation of 1 (V/V·min), to obtain the silicate bacteria fermentation broth.

The present invention further provides a soil-structure improving bio-organic fertilizer, which has a total content of viable bacteria up to 400-1500 million cfu/g.

Preferably, the content of viable Bacillus subtilis is 380-1400 million cfu/g; and the content of viable silicate bacteria is 20-100 million cfu/g.

According to the above technical solution, compared with the prior art, the present invention has the following beneficial effects:

(1) In the present invention, organic wastes such as cow dung are aerobically fermented; Bacillus subtilis and silicate bacteria are inoculated; and two microorganisms and a solid substrate are synergistically fermented by solid-state fermentation. On the one hand, cow dung with higher cellulose content is taken as the main material, and plant straws are added, to increase the content of cellulose in the aerobic fermentation substrate, improve the soil structure improvement function of the present invention, and solve the problem of recycling of the crop straws. On the other hand, Bacillus subtilis plays a role of promoting the growth and reproduction of silicate bacteria, to further increase the number and viability of strains, so that the content of viable microbial bacteria reaches 400-1500 million cfu/g, the content of viable Bacillus subtilis is 380-1400 million cfu/g, and the content of viable silicate bacteria is 20-100 million/g, thereby providing a guarantee for the strains to solubilize phosphorus and potassium and exert the disease resistance potential.

(2) Silicate bacteria can produce a large number of polysaccharides, which is conducive for the soil to form an aggregate structure, thereby effectively improving the soil structure.

(3) At the same time, the microorganisms and the solid substrate are co-fermented by solid-state fermentation, so that metabolites of the strains are all present in the solid medium. Compared with the prior art that the strains are absorbed into the solid substrate according to a certain proportion after liquid fermentation, and most of the metabolites of the strains are dissolved in the liquid medium, but not all of the metabolites are utilized, the strains and the solid fermentation substrate are organically combined in the present invention so that the obtained microbial fertilizer has stable fertilizer effect, increases the utilization rate of the metabolites of the strains, improves the soil, improves the ability of crops to absorb soil nutrients, increases the yield of agricultural products, and improves the quality of agricultural products.

### DETAILED DESCRIPTION OF THE EMBODIMENTS

The technical solutions in the embodiment of the present invention will be clearly and fully described below. Apparently, the described embodiment is merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1

Preparation of Bacillus subtilis fermentation broth: 150 ml of culture medium was injected into a 500 ml shake flask; Bacillus subtilis strains were transferred into a shake flask with 5 ml of sterile water, and cultured for 20 hours at 28-30° C. and 200 rpm to obtain Bacillus subtilis broth; and the Bacillus subtilis broth was inoculated into a seed tank according to an inoculation amount of 3% of the weight of a seed tank culture medium, then mixed and fermented, and cultured for 24 hours at 30° C. and 190 rpm, with a ventilation of (V/V·min), to obtain the silicate bacteria fermentation broth.

1. The shake flask culture medium includes:

200 g of potato (peeled and cooked), 20 g of sucrose (white sugar or glucose) and 1000 ml of distilled water, with a pH of 6.5.

2. The seed tank culture medium includes:

0.2% of ammonium sulfate, 0.286% of glucose, 0.714% of potato starch, 0.030% of dipotassium hydrogen phosphate, 0.020% of sodium chloride, 0.020% of potassium chloride, of magnesium sulfate, 0.003% of manganese sulfate, 0.003% of ferrous sulfate, of yeast powder and 98.344% of distilled water.

Preparation of silicate bacteria fermentation broth: 150 ml of culture medium was injected into a 500 ml shake flask; silicate bacteria strains were transferred into a shake flask with 5 ml of sterile water, and cultured for 30 hours at 28-30° C. and 200 rpm to obtain silicate bacteria broth; and the silicate bacteria broth was inoculated into a seed tank according to an inoculation amount of 3% of the weight of a seed tank culture medium, then mixed and fermented, and cultured for 30 hours at 30° C. and 200 rpm, with a ventilation of 1 (V/V·min), to obtain the silicate bacteria fermentation broth.

1. The shake flask culture medium includes: 5 g of sucrose, 0.8 g of yeast extract, g of ammonium sulfate, 0.8 g of magnesium sulfate, 1 g of K₂HPO₄, 0.1 g of sodium chloride and 1 g of calcium carbonate, with a pH of 7.0.

2. The seed tank culture medium includes: 0.5% of soybean cake powder, 0.5% of sucrose, 0.5% of yeast powder, 0.01% of ammonium sulfate, 0.08% of magnesium sulfate, 0.1% of dipotassium hydrogen phosphate, 0.01% of sodium chloride, 0.1% of calcium carbonate, 0.001% of ferrous sulfate and 98.199% of distilled water.

### Embodiment 2

45 parts of cow dung, 20 parts of tomato straw, 15 parts of corn straw, 20 parts of moisture regulating auxiliary materials and 0.5% of commercially available decomposing agent (a CM decomposing agent of Shandong Yian Biotechnology Co., Ltd. was used, and the mass ratio of the CM decomposing agent to the fermentation materials was 0.5%) were taken, and then stacked according to the length×width×height of 50 m×5 m×1.5 m for aerobic fermentation. The fermentation materials with an initial moisture content of 50-55% were turned over with a turner for 3-4 times in the whole aerobic fermentation period. When the temperature of a pile was higher than 65° C., the temperature was reduced by turning over, stirring and aeration. When the temperature of the pile was higher than 55° C., the fermentation was continued for 7 days, to obtain a solid fermentation substrate after the fermentation was finished. The technical indexes of the weight percentage of the solid fermentation substrate include: 32% of moisture content, 46% of organic matter, 1.25% of total nitrogen, 1.2% of P₂O₅ and 1.7% of K₂O.

The Bacillus subtilis fermentation broth prepared in embodiment 1 at the inoculation amount of 5% of the mass of the solid medium and the silicate bacteria fermentation broth at the inoculation amount of 10% of the mass of the solid medium were mixed uniformly, and then inoculated into the solid fermentation substrate for fermentation. The fermentation materials were stacked according to the length×width×height of 50 m×5 m×0.5 m. The raw materials with an initial moisture content of 47% were fermented at 35° C., turned over every 24 hours, and fermented for 7 days to obtain a product 1 of bio-organic fertilizer.

The bacterial content and the nutrient index of the obtained product were detected. The results are shown as follows:

Product 1 has a living bacteria count of 1 billion cfu/g, including 950 million cfu/g of Bacillus subtilis and 50 million cfu/g of silicate bacteria, the number of mold and miscellaneous bacteria of ≤3.0×10⁶ cfu/g, a moisture content of ≤30%, a fineness of ≥80%, a pH of 7.5, a shelf life of 12 months, a dosage form of powder, and a content of organic matter of ≥40%.

Two kinds of seed tank fermentation broth in embodiment 1 at both of the inoculation amount of 8% were uniformly mixed with the aerobic fermentation substrate of cow dung in embodiment 2 for solid-state fermentation. The fermentation materials were stacked according to the length×width×height of 50 m×5 m×0.5 m. The raw materials with an initial moisture content of 47% were fermented at 35° C., turned over every 24 hours, fermented for 7 days, and then extruded and granulated to obtain a product 2 of bio-organic fertilizer.

The bacterial content and the nutrient index of the obtained product were detected. The results are shown as follows:

Product 2 has the living bacteria count of 500 million cfu/g, including 480 million cfu/g of Bacillus subtilis and 20 million cfu/g of silicate bacteria, the number of mold and miscellaneous bacteria of ≤3.0×10⁶ cfu/g, the moisture content of ≤15%, the fineness of ≥80%, the pH of 7.5, the shelf life of 12 months, the dosage form of granule, and the content of organic matter of ≥40%.

### Embodiment 3

55 parts of cow dung, 40 parts of tomato straw, 5 parts of moisture regulating auxiliary materials and 0.5% of commercially available decomposing agent (the CM decomposing agent of Shandong Yian Biotechnology Co., Ltd. was used, and the mass ratio of the CM decomposing agent to the fermentation materials was 0.5%) were taken, and then stacked according to the length×width×height of 50 m×5 m×1.5 m for aerobic fermentation. The fermentation materials with an initial moisture content of 50% were turned over with the turner for 3-4 times in the whole aerobic fermentation period. When the temperature of the pile was higher than 65° C., the temperature was reduced by turning over, stirring and aeration. When the temperature of the pile was higher than 55° C., the fermentation was continued for 7 days, to obtain a solid fermentation substrate after the fermentation was finished. The technical indexes of the weight percentage of the solid fermentation substrate include: 35% of moisture content, 40% of organic matter, 1.3% of total nitrogen, 1.3% of P₂O₅ and 1.6% of K₂O.

The Bacillus subtilis fermentation broth prepared in embodiment 1 at the inoculation amount of 5% of the mass of the solid medium and the silicate bacteria fermentation broth at the inoculation amount of 10% of the mass of the solid medium were mixed uniformly, and then inoculated into the solid fermentation substrate for fermentation. The fermentation materials were stacked according to the length×width×height of 50 m×5 m×0.5 m. The raw materials with an initial moisture content of 47% were fermented at 35° C., turned over every 22 hours, and fermented for 7 days to obtain a bio-organic fertilizer.

### Embodiment 4

55 parts of cow dung, 40 parts of corn straw, 5 parts of moisture regulating auxiliary materials and 0.5% of commercially available decomposing agent (the CM decomposing agent of Shandong Yian Biotechnology Co., Ltd. was used, and the mass ratio of the CM decomposing agent to the fermentation materials was 0.5%) were taken, and then stacked according to the length×width×height of 50 m×5 m×1.5 m for aerobic fermentation. The fermentation materials with an initial moisture content of 55% were turned over with the turner for 3-4 times in the whole aerobic fermentation period. When the temperature of the pile was higher than 65° C., the temperature was reduced by turning over, stirring and aeration. When the temperature of the pile was higher than 55° C., the fermentation was continued for 10 days, to obtain a solid fermentation substrate after the fermentation was finished. The technical indexes of the weight percentage of the solid fermentation substrate include: 30% of moisture content, 45% of organic matter, 1.2% of total nitrogen, 1.2% of P₂O₅ and 1.5% of K₂O.

The seed tank fermentation broth of Bacillus subtilis prepared in embodiment 1 at the inoculation amount of 5% of the mass of the solid medium and the seed tank fermentation broth of silicate bacteria at the inoculation amount of 15% of the mass of the solid medium were mixed uniformly, and then inoculated into the solid fermentation substrate for fermentation. The fermentation materials were stacked according to the length×width×height of 50 m×5 m×0.5 m. The raw materials with an initial moisture content of 50% were fermented at 35° C., turned over every 26 hours, and fermented for 5 days to obtain a bio-organic fertilizer.

The microbial strains used in embodiments 2-4 include: Bacillus Subtilis with an accession number of ACCC 19742 in Agricultural Culture Collection of China, and silicate bacteria also known as Bacillus mucilaginosus with an accession number of ACCC10013 in Agricultural Culture Collection of China.

### Embodiment 5

Field Test

To verify the application effect of the functional bio-organic fertilizer obtained by the production method of multi-strain solid synergistic fermentation according to the present invention, the inventor conducted a four-season positioning test for improving saline-alkali soil from 2018 to 2020. The bio-organic fertilizer prepared in embodiment 2 of the present invention was taken as a test group; and the conventional fertilization moeds of farmers and commercially available similar microbial organic fertilizers were taken as a control group.

Test crops of the present invention include winter wheat (Linmai 4) and corn (Shanda 1). For the winter wheat test in the first season, fertilizing, plowing and sowing were conducted on Oct. 8, 2018; and harvesting was completed in early June 2019. For the corn test in the second season, fertilizing, plowing and sowing were conducted on Jun. 18, 2019; and harvesting was completed in early October. For the winter wheat test in the third season, fertilizing, plowing and sowing were conducted on Oct. 15, 2019; and harvesting was completed in early June 2020. For the corn test in the fourth season, fertilizing, plowing and sowing were conducted on Jun. 20, 2020; and harvesting was completed in early October. The sowing density of corn was 4000 plants/mu; the sowing quantity of wheat per mu was 18 kg; when harvesting in every season, the crops were harvested, weighed and calculated according to the plot; and other cultivation and management measures were consistent with those of local farmers.

Conventional fertilization modes of farmers: (converted) 16 kg of nitrogen fertilizer, 10 kg of phosphorus fertilizer and 6 kg of potassium fertilizer per mu were applied in the wheat season; (converted) 12 kg of nitrogen fertilizer, 8 kg of phosphorus fertilizer and 7 kg of potassium fertilizer per mu were applied in the corn season; the fertilizers were spread over the soil; and then the soil was ploughed.

Commercially available similar bio-organic fertilizer has the living bacteria count (cfu) of ≥1 billion cfu/g, microbial strains including Bacillus megatherium and Bacillus Subtilis, and the content of organic matter of ≥40%.

The product 1 of bio-organic fertilizer prepared in embodiment 2 of the present invention was taken as the test group.

Fertilization modes of commercially available similar product and the test group of the present invention: on the basis of optimized fertilization, 150 kg/mu of base fertilizer was applied; the optimized fertilization mode includes: (converted) 15 kg of nitrogen fertilizer, 8 kg of phosphorus fertilizer and 4 kg of potassium fertilizer were applied in the wheat season; (converted) 12 kg of nitrogen fertilizer, 8 kg of phosphorus fertilizer and 7 kg of potassium fertilizer were applied in the corn season; the fertilizers were spread over the soil; and then the soil was ploughed. Effects of different treatments on physical and chemical properties of soil after the end of the test in October 2020 are shown as follows:

TABLE 1
Effects of Different Treatments on Bulk Density and pH of Soil

| | Bulk density after harvest of corn in 2020 | | | | | | | |
|---|---|---|---|---|---|---|---|---|
| | (g/cm³) | | | | pH of soil after harvest of corn in 2020 | | | |
| Treatment | Repeat 1 | Repeat 2 | Repeat 3 | Mean | Repeat 1 | Repeat 2 | Repeat 3 | Mean |
| Conventional | 1.280 | 1.255 | 1.29 | 1.275a | 8.0 | 8.05 | 7.98 | 8.01a |
| method | | | | | | | | |
| Commercially | 1.21 | 1.235 | 1.25 | 1.23a | 7.95 | 7.96 | 7.94 | 7.95a |
| available similar | | | | | | | | |
| product | | | | | | | | |
| Product of the | 1.15 | 1.17 | 1.17 | 1.16b | 7.85 | 7.80 | 7.84 | 7.83b |
| present invention | | | | | | | | |

Table 1 shows that the bulk density of soil after application of the commercially available similar product is reduced by 3.53% in mean in comparison with the conventional control group, and the bulk density of soil after application of the product of the present invention is reduced by 9.02% in comparison with the conventional control group, which indicates that the application of the product of the present invention has significant advantages in loosening soil, increasing soil porosity and enhancing permeability in comparison with the conventional control group and the control group of commercially available similar product, and promotes the exchange of water, gas and heat in the soil and the activities of microorganisms, thereby providing excellent soil conditions for crop roots to absorb nutrients and water. On the other hand, the product of the present invention has more apparent pH improvement effect than that of the commercially available similar product and the conventional control group.

TABLE 2
Effects of Different Treatments on Particle Size
Classification of Water-stable Aggregates of Soil

| | >1-3 | >0.5-1 | >0.25-0.5 |
|---|---|---|---|
| Treatment | mm | mm | mm |
| Conventional method | 4.38bc | 7.53ab | 8.68bc |
| Commercially available similar product | 5.03ab | 7.25b | 9.14b |
| Product of the present invention | 5.16a | 8.07a | 9.87a |

Soil aggregates, as good soil structures, play an important role in regulating soil water, air and temperature. In saline-alkali soil, the low content of soil aggregates greater than mm is a main factor that affects the growth of plants. Table 2 shows that after application of the product of the present invention, the aggregates greater than 0.25 mm in the soil are on an increasing trend. In the particle sizes of >0.25-0.5 mm, >0.5-1 mm and >1-3 mm, the particle size is significantly increased after application of the product of the present invention in comparison with that after application of the conventional and commercially available similar products. Such products enhance the stability of soil micro-aggregates in water. In the classification of >0.5-1 mm and >0.25-0.5 mm, the content of water-stable aggregates after the two treatments is apparently higher than that after the conventional treatment, with a very significant difference; and the effect of the product of the present invention is better than that of commercially available similar product.

TABLE 3
Effects of Different Treatments on Crop Yield

| | Yield of wheat in 2020 (kg/mu) | | | | Yield of corn in 2020 (kg/mu) | | | |
|---|---|---|---|---|---|---|---|---|
| Treatment | Repeat 1 | Repeat 2 | Repeat 3 | Mean | Repeat 1 | Repeat 2 | Repeat 3 | Mean |
| Conventional | 393.4 | 386.2 | 388.2 | 385.1b | 423.7 | 421.8 | 441.6 | 429.0c |
| method | | | | | | | | |
| Commercially | 388.5 | 408.4 | 399.5 | 401.7b | 462.7 | 458.4 | 440.9 | 454.0b |
| available similar | | | | | | | | |
| product | | | | | | | | |
| Product of the | 411.4 | 417.8 | 417.6 | 423.7a | 491.5 | 482.3 | 477.6 | 483.8a |
| present invention | | | | | | | | |

Table 3 shows that, the yield of wheat after application of the product of the present invention is increased by 22 kg, with an increase of 5.48% compared with that after application of the commercially available similar products, and with an increase of 10.02% compared with that after the conventional control treatment, so the effect is significant; and the yield of corn after application of the product of the present invention is increased by 29.8 kg, with an increase of 6.56% compared with that after application of the commercially available similar products, and is increased by 12.77% compared with that after the conventional control treatment, so the effect is significant.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A preparation method of a soil-structure improving bio-organic fertilizer, comprising the following steps:
S 1: aerobically fermenting cow dung-based organic materials to obtain a solid-state fermentation medium;
S2: mixing and inoculating Bacillus subtilis fermentation broth and silicate bacteria fermentation broth into the solid-state fermentation medium obtained in S1 for solid-state fermentation;
S3: after the fermentation is finished, extruding and granulating or directly sieving to prepare the soil-structure improving bio-organic fertilizer;
the cow dung-based organic materials in step S1 are composed of any of the following three types in parts by weight:
1) 50-60 parts of cow dung, 40-50 parts of tomato straw and 5-10 parts of moisture regulating auxiliary materials;
2) 50-60 parts of cow dung, 40-50 parts of corn straw and 5-10 parts of moisture regulating auxiliary materials;
3) 40-50 parts of cow dung, 20-30 parts of tomato straw, 15-20 parts of corn straw and 5-10 parts of moisture regulating auxiliary materials.

2. The preparation method of the soil-structure improving bio-organic fertilizer according to claim 1, wherein in aerobic fermentation of step S1: the cow dung-based organic materials has an initial moisture content of 50-55%; and after the temperature of a pile reaches 55° C., the fermentation is continued for 7-10 days, and then the fermentation is finished.

3. The preparation method of the soil-structure improving bio-organic fertilizer according to claim 1 wherein in aerobic fermentation of step S1: the cow dung-based organic materials are stacked according to the length×width×height of 50 m×5 m×1.5 m; during the whole period of aerobic fermentation, the pile is turned over for 3-4 times; and when the temperature of the pile is higher than 65° C., the temperature is lowered by turning over, stirring and aeration.

4. The preparation method of the soil-structure improving bio-organic fertilizer according to claim 1, wherein the inoculation weights of the Bacillus subtilis fermentation broth and the silicate bacteria fermentation broth to the solid-state fermentation medium in step S2 are of the weight of the solid-state fermentation medium, respectively.

5. The preparation method of the soil-structure improving bio-organic fertilizer according to claim 1, wherein in the solid-state fermentation culture of step S2: the solid fermentation materials have an initial moisture content of 40-50%; the pile is turned over every 22-26 hours during fermentation; and the fermentation time is 5-7 days.

6. The preparation method of the soil-structure improving bio-organic fertilizer according to claim 1, wherein in the solid-state fermentation culture of step S2: the solid fermentation materials are stacked according to the length×width×height of 50 m×5 m×0.5 m; and the solid fermentation materials have a fineness of 70-100 meshes, a C/N ratio of 25-30, and a pH of 6.5-7.5.

7. The preparation method of the soil-structure improving bio-organic fertilizer according to claim 6, wherein in the solid-state fermentation culture of step S2: the solid fermentation materials have the fineness of 80 meshes, the C/N ratio of 28, and the pH of 7.

8. The preparation method of the soil-structure improving bio-organic fertilizer according to claim 1, wherein a preparation method of the Bacillus subtilis fermentation broth in step S2 comprises: inoculating the Bacillus subtilis broth obtained by shake-flask culture into a seed tank culture medium according to an inoculation amount of 2-5% of the mass of the seed tank culture medium, and culturing for 22-26 hours at 28-32° C. and 180-200 rpm, with a ventilation of 0.9 (V/V·min), to obtain the Bacillus subtilis fermentation broth;
a preparation method of the silicate bacteria fermentation broth comprises: inoculating the silicate bacteria broth obtained by shake-flask culture into a seed tank culture medium according to the inoculation amount of 4-6% of the mass of the seed tank culture medium, and culturing for 28-32 hours at 28-32° C. and 180-220 rpm, with a ventilation of 1 (V/V·min), to obtain the silicate bacteria fermentation broth.

9. A soil-structure improving bio-organic fertilizer prepared according to claim 1, having a total content of viable bacteria up to 400-1500 million cfu/g.

10. The soil-structure improving bio-organic fertilizer according to claim 9, wherein the content of viable Bacillus subtilis is 380-1400 million cfu/g; and the content of viable silicate bacteria is 20-100 million cfu/g.
